(19) United States
(12) Patent Application Publication
(10) Pub. No.: US 2026/0048280 A1
(43) Pub. Date: Feb. 19, 2026

(54) HISTOTRIPSY SYSTEMS AND METHODS

(71) Applicant: HISTOSONICS, INC., Plymouth, MN (US)

(72) Inventor: Ryan MILLER, Saline, MI (US)

(21) Appl. No.: 19/103,752

(22) PCT Filed: Aug. 17, 2023
(86) PCT No.: PCT/US2023/072412

§ 371 Date: Feb. 13, 2025

Related U.S. Application Data
(60) Provisional application No. 63/371,728, filed on Aug. 17, 2022.

(51) Int. Cl. A61N 7/00

(52) U.S. Cl. CPC A61N 7/00; A61N 2007/0039

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. The histotripsy therapy system can include a robotic positioning system configured to move and translate an ultrasound transducer array through a treatment volume.

### PRIORITY CLAIM

This patent application claims priority to U.S. provisional patent application No. 63/371,728, titled “HISTOTRIPSY SYSTEMS AND METHODS”, and filed on Aug. 17, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU), cryo, or radiation, Histotripsy relies on the mechanical action of cavitation for tissue destruction and not on heat, cold or ionizing energy.

### SUMMARY

An ultrasound method is provided, comprising moving an ultrasound transducer array of an ultrasound system along an approach plane to place a focus of the ultrasound transducer array on a target treatment location within a treatment volume of a subject; determining an aperture width of the ultrasound transducer array in the approach plane at a depth of an acoustic window; and determining a tilt angle of the ultrasound transducer array that centers an acoustic beam of the ultrasound transducer array on the acoustic window while placing the focus of the ultrasound transducer array on the target treatment location.

In some aspects, determining the aperture width further comprises calculating a beam angle of the ultrasound transducer array in the approach plane.

In one aspect, determining the beam angle is based on the specific ultrasound transducer geometry.

In some aspects, the ultrasound transducer geometry comprises a circular aperture.

In one aspect, the ultrasound transducer geometry comprises an ellipsoidal aperture.

In some aspects, the ultrasound transducer geometry comprises a cropped elliptical shape.

In one aspect, the ultrasound transducer geometry comprises cropped elliptical regions, spherical regions, and fillet regions that transition between the cropped elliptical regions and the spherical regions.

In another aspect, the approach plane is rotated around a z-axis of the transducer array.

In some aspects, the tilt angle is out of the z-axis.

In one aspect, moving the ultrasound transducer array further comprises robotically moving the ultrasound transducer array with a robotic positioning system.

In one aspect, the method includes moving the ultrasound transducer array to the tilt angle.

In another aspect, the method includes applying ultrasound energy to the target treatment location.

In some aspects, applying ultrasound energy comprises applying histotripsy energy to generate cavitation at the treatment location.

In one aspect, the depth is user-selected.

In another aspect, the depth is automatically selected.

In some aspects, the depth is a distance from the target treatment location to an obstruction.

In some aspects, the obstruction comprises one or more bones in the subject.

In some aspects, the one or more bones define the acoustic window.

In one aspect, the tilt angle reduces or eliminates obstruction of at least a portion of a transmit aperture of the ultrasound transducer array.

In some aspects, the tilt angle is determined with one or more processors of the ultrasound system.

A method of providing ultrasound therapy is provided, comprising moving an ultrasound transducer array of an ultrasound system along an approach plane to place a focus of the ultrasound transducer array on a target treatment location within a treatment volume of a subject; determining an aperture width of the ultrasound transducer array in the approach plane at a depth of an acoustic window; determining a tilt angle that centers an acoustic beam of the ultrasound transducer array on the selected acoustic window while placing the focus of the ultrasound transducer array on the target treatment location; controlling movement of the ultrasound transducer with a robotic positioning system along the approach plane to assume the tilt angle that places the focus on the target treatment location and centers the acoustic beam on the selected acoustic window; and delivering ultrasound therapy to the target treatment location with the ultrasound transducer array.

In some aspects, the ultrasound therapy comprises histotripsy therapy.

In one aspect, delivering ultrasound therapy further comprises generating cavitation at the target treatment location.

In some aspects, determining the aperture width further comprises calculating a beam angle of the ultrasound transducer array in the approach plane.

In another aspect, determining the beam angle is based on the specific ultrasound transducer geometry.

In some aspects, the ultrasound transducer geometry comprises a circular aperture.

In one aspect, the ultrasound transducer geometry comprises an ellipsoidal aperture.

In another aspect, the ultrasound transducer geometry comprises a cropped elliptical shape.

In some aspects, the ultrasound transducer geometry comprises cropped elliptical regions, spherical regions, and fillet regions that transition between the cropped elliptical regions and the spherical regions.

In one aspect, the approach plane is rotated around a z-axis of the transducer array.

In another aspect, the depth is user-selected.

In some aspects, the depth is automatically selected.

In one aspect, the depth is a distance from the target treatment location to an obstruction.

In another aspect, the obstruction comprises one or more bones in the subject.

In some aspects, the one or more bones define the acoustic window.

In some aspects, the tilt angle reduces or eliminates obstruction of at least a portion of a transmit aperture of the ultrasound transducer array.

In some aspects, the tilt angle is determined with one or more processors of the ultrasound system.

A method of delivering ultrasound energy to tissue is provided, comprising moving an ultrasound transducer array of an ultrasound system along an approach plane to place a focus of the ultrasound transducer array on a target treatment location within a treatment volume of a subject; detecting an obstruction in the subject that blocks at least a portion of a transmit aperture of the ultrasound transducer array; identifying an acoustic window that minimizes or eliminates an amount of the transmit aperture that is blocked; determining a tilt angle that centers an acoustic beam of the ultrasound transducer array on the acoustic window while placing a focus of the ultrasound transducer array on the target treatment location; controlling a robotic positioning system to orient the ultrasound transducer array along the tilt angle while maintaining the focus at the target treatment location; and delivering ultrasound therapy to the target treatment location with the ultrasound transducer array.

In some aspects, the ultrasound therapy comprises histotripsy therapy.

In one aspect, delivering ultrasound therapy further comprises generating cavitation at the target treatment location.

In some aspects, the tissue volume comprises a tumor.

In one aspect, the obstruction is selected from the group consisting of bones, hard tissues, mineral deposits, implanted medical devices, and tissue implants within the subject.

In another aspect, determining the tilt angle is based on the depth of the obstruction.

In some aspects, the depth is user-selected.

In one aspect, the depth is automatically selected.

In one aspect, the depth is a distance from the target treatment location to an obstruction.

In some aspects, the obstruction defines the acoustic window.

In one aspect, the tilt angle reduces or eliminates obstruction of at least a portion of a transmit aperture of the ultrasound transducer array.

In some aspects, the tilt angle is determined with one or more processors of the ultrasound system.

An ultrasound system, comprising: a robotic positioning system; an ultrasound transducer array disposed on the robotic positioning system, the ultrasound transducer array being configured to deliver ultrasound pulses into a subject; one or more processors operatively coupled to the robotic positioning system and the ultrasound transducer array; a non-transitory computing device readable medium having instructions stored thereon for generating a treatment plan for ultrasound therapy, wherein the instructions are executable by the one or more processors to cause the ultrasound system to: move the ultrasound transducer array along an approach plane to place a focus of the ultrasound transducer array on a target treatment location within a treatment volume of the subject; determine an aperture width of the ultrasound transducer array in the approach plane at a depth of an acoustic window; and determine a tilt angle of the ultrasound transducer array that centers an acoustic beam of the ultrasound transducer array on the acoustic window while placing the focus of the ultrasound transducer array on the target treatment location.

In one aspect, the instructions are further configured to cause the ultrasound system to: control the robotic positioning system to orient the ultrasound transducer array at the tilt angle while maintaining the focus at the target treatment location; and deliver ultrasound therapy to the target treatment location with the ultrasound transducer array.

In another aspect, the ultrasound system is configured to determine the aperture width by calculating a beam angle of the ultrasound transducer array in the approach plane.

In some aspects, determining the beam angle is based on a specific ultrasound transducer geometry.

In another aspect, the ultrasound transducer geometry comprises a circular aperture.

In one aspect, the ultrasound transducer geometry comprises an ellipsoidal aperture.

In another aspect, the ultrasound transducer geometry comprises a cropped elliptical shape.

In some aspects, the ultrasound transducer geometry comprises cropped elliptical regions, spherical regions, and fillet regions that transition between the cropped elliptical regions and the spherical regions.

In other aspects, the approach plane is rotated around a z-axis of the transducer array.

In one aspect, the tilt angle is out of the z-axis.

In another aspect, the ultrasound therapy comprises histotripsy therapy.

In one aspect, the depth is user-selected.

In one aspect, the system includes an input device configured to receive the user-selected depth from a user. In some aspects, the input device comprises a graphical user interface.

In one aspect, the depth is automatically determined by the one or more processors.

In another aspect, the depth is automatically determined based on pre-treatment medical imaging.

In one aspect, the depth is a distance from the target treatment location to an obstruction.

In one aspect, the obstruction comprises one or more bones in the subject.

In another aspect, the one or more bones define the acoustic window.

In some aspects, the tilt angle reduces or eliminates obstruction of at least a portion of a transmit aperture of the ultrasound transducer array.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is one embodiment of a histotripsy therapy and imaging system with a coupling system.

FIGS. 3A-3B illustrate one example of translating a transducer array towards a blockage.

FIG. 4 is an example of tilting and translating a transducer array to avoid or reduce the effects of a blockage.

FIGS. 5A-5D illustrate schematic drawings and equations determining a new approach plane, a beam angle, and a tilt angle of a transducer array.

FIG. 6 is a flowchart showing a method of delivering ultrasound to tissue.

FIGS. 7A-7E show additional embodiments on how adjusting a depth of the acoustic window changes the path through which the ultrasound transducer array navigates the tissue volume.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system/histotripsy system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

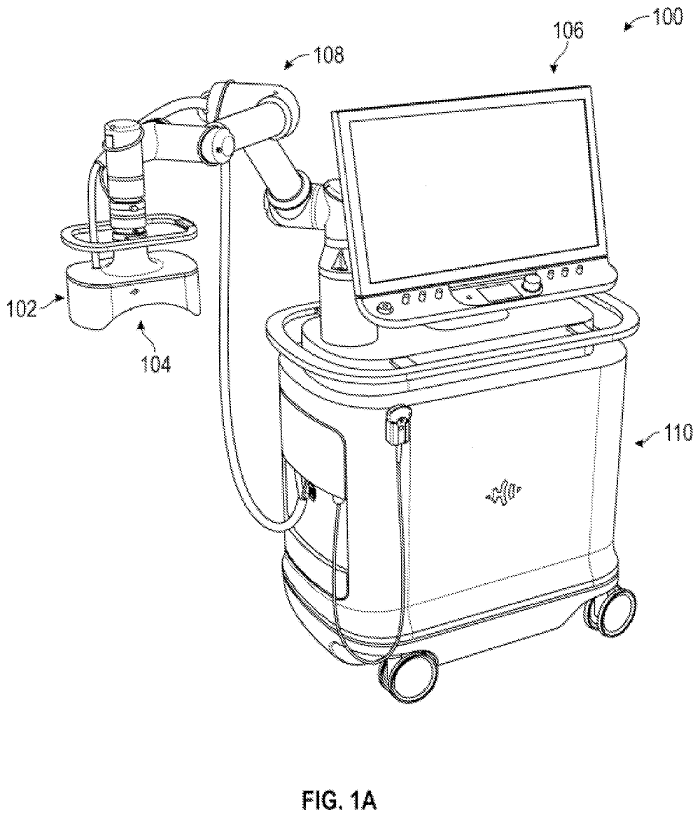

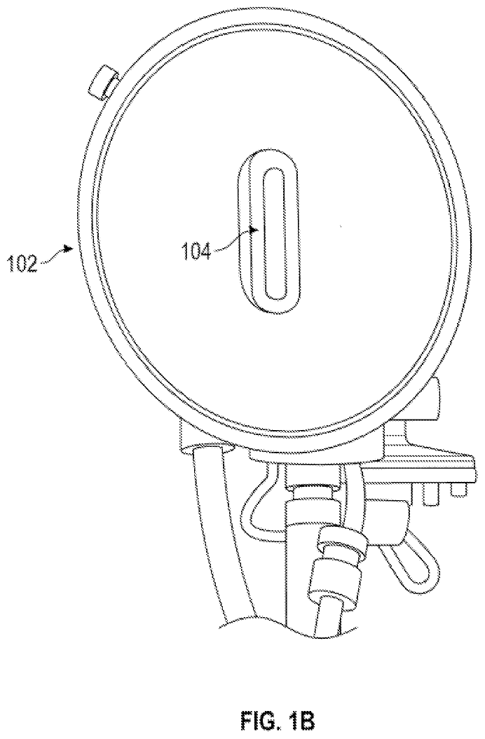

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., fluidics cart, anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to, fusion with, and display of patient medical data including but not limited to laboratory and historical medical record data. The Cart may be configured work with cloud-based data management systems or electronic patient records and pull or stream data real-time, in advance of or after histotripsy procedures.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ˜24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer to the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue or acoustic coupling to tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses of less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other such systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, cone beam CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

The system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking, speed and velocity.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degrees of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Robotic Positioning to Avoid Obstructions

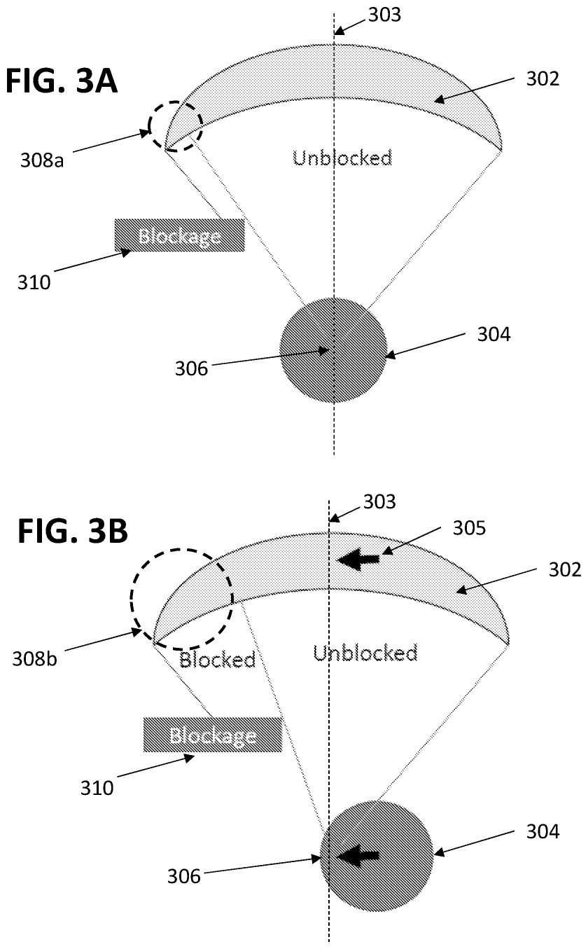

A transducer array of the present disclosure can include a plurality of transducer elements configured to transmit ultrasound energy into tissue. Generally, a transmit aperture can be defined as the portion(s) of the transducer array that are actively transmitting ultrasound energy towards a focus of the transducer array. In some examples, referring to FIGS. 3A-3B, bones or other aberrators (e.g., ribs, skull, bones, implantable medical devices, tissue implants, other hard tissues etc.) positioned between a therapy transducer array 302 and a target tissue volume 304 can block or prevent some or all of transducer elements or a portion of the transmit aperture from directing ultrasound waves to the focus 306. In FIG. 3A, the focus 306 is positioned in a central portion of the target tissue volume 304 along a pose axis 303, with a relatively small subset 308a of transducer elements or the transducer aperture being blocked from transmitting ultrasound waves to the focus by an aberrator or blockage 310. Generally the transducer and robotic positioning system are positioned over the patient, so in FIG. 3A, the illustrated pose axis 303 can be generally aligned with a Z axis that extends out of the patient and in line with or through the transducer array. While the therapy array can still be configured to generate cavitation with some of the transducer elements or transmit aperture blocked, the array may require a higher driving voltage to achieve cavitation/bubble cloud initiation at the focus.

As is described herein, in some embodiments a treatment plan is implemented which divides the target tissue volume into a plurality of discrete treatment locations and provides a pathway for mechanical steering/movement/motion (e.g., automated robotic movement) of the transducer array focus through the discrete treatment locations to provide therapy to the entire tissue volume. While movement through the treatment plan is generally described as being controlled by the robotic positioning system, it should also be understood that in some embodiments, electronic steering of the transducer array can be used to make fine adjustments to the bubble cloud position (e.g., in the x, y, and/or z directions). Therefore, embodiments are considered in which navigation through the treatment plan is accomplished with some combination of mechanical/robotic steering and electronic steering of the transducer focus.

To fully treat a given target tissue volume, the treatment plan typically requires positioning the transducer focus at the lateral extremes of the target tissue volume. In some examples, this is achieved by robotically or mechanically moving the therapy transducer laterally. Referring to FIG. 3B, the therapy transducer array 302 has been robotically moved laterally in direction 305 to position the focus 306 at the lateral extremes of the target tissue volume, causing a larger subset 308b of the transducer elements or transmit aperture to be blocked by the aberrator or blockage 310. It can be seen in FIG. 3B that while the therapy transducer array has been moved laterally, it maintains the same pose axis 303 as in FIG. 3A. As shown in FIG. 3B, in order to move the focus 306 to the far extreme of the target tissue volume (e.g., the left most extreme on the page), the therapy transducer array is also translated in the same direction (e.g., to the left of the page). Thus, the motion (e.g., robotic or mechanical movement) of the therapy transducer array to achieve treatment at a given point in a target treatment volume according to the technique of FIGS. 3A-3B can include: (1) maintaining a constant pose axis (e.g., parallel to the original pose axis or Z-axis used to define the treatment plan), and (2) movement to a given treatment point is in the direction of that treatment point (again, relative to the original pose at the center used to define the treatment plan). The increased blockage from traversing the therapy transducer array to lateral extremes of the target tissue volume can result in significantly increased driving voltage being required to generate a bubble cloud at the focus 306. In some examples, these increased driving voltages can result in increased pre-focal tissue heating, which may require additional cooling periods to be added to the treatment plan.

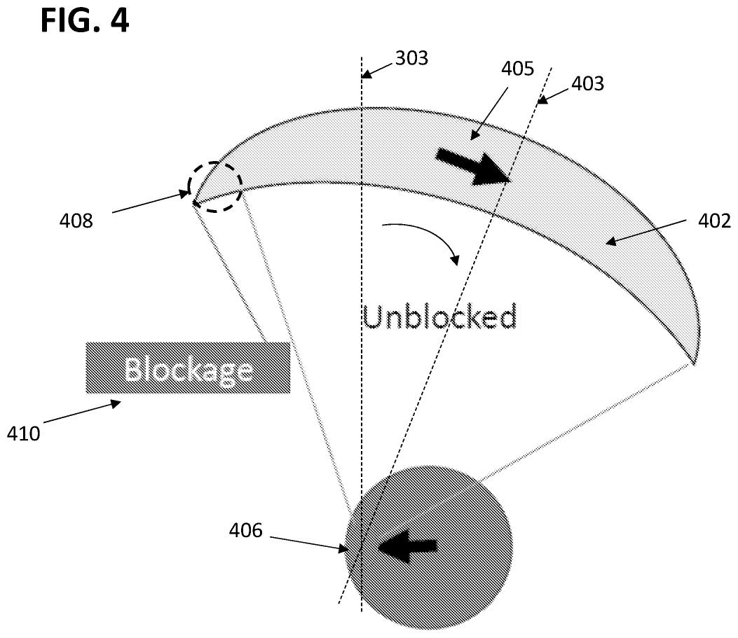

According to one aspect of the disclosure, referring to FIG. 4, instead of simply translating the therapy transducer array to position the focus at a given treatment location in the volume that may result in increased blockage of some or all of the therapy transducer array (e.g., as shown in FIG. 3B), processors and/or electronic controllers of the system and/or the robotic positioning system/arm can be configured to translate and tilt the therapy transducer array 402 to avoid the blockage or reduce the amount of blockage of a subset 408 of the transmit aperture, while maintaining a similar acoustic window access to the treatment location at the periphery of the target tissue volume. The resulting translation in direction 405 and tilting of the therapy transducer array (tilting from pose axis 303 to pose axis 403) can minimize the blockage or completely avoid the blockage and potentially reduce or eliminate the increased driving voltage that would have been necessary to treat this location within the target tissue volume using the standard approach (as in FIG. 3B). Put another way, the transducer array is tilted so that the pose axis 403 of the transducer array is no longer aligned with the Z axis extending up and out of the patient (e.g., axis 303). In particular, pose axis 403 is off axis or disposed at an angle relative to the Z axis or the axis used during formation of the treatment plan.

It should be noted that, in contrast to the movement in the embodiment of FIG. 3B, in the embodiment of FIG. 4, the transducer array is translated in the opposite direction as the desired movement and placement of the focus. For example, in FIG. 3B, to move the focus to the left most extreme of the target tissue volume (relative to the page), the transducer array is also moved in the left-direction (relative to the page). However, in FIG. 4, the transducer array is translated in the opposite direction of the desired movement of the focus. Specifically, to move the focus to the left most extreme of the target tissue volume, the transducer array is translated in the right-direction (relative to the page) and then tilted (e.g., in the clockwise direction relative to the page) to maintain a similar acoustic window to the treatment location. The transducer array may be generally translated in a generally opposite or about 180 degrees apart/opposite to the (intended) focus in an X, Y, and Z coordinate system/plane, and then tilted to maintain a similar acoustic window to the treatment location.

During therapy according to a treatment plan, the robotic arm may move the transducer array to the plurality of discrete treatment locations. In some situations, one or more of the treatment locations may result in the transducer array being translated into a position where a blockage or aberrator blocks one or more therapy transducers or a subset or portion of the transmit aperture. This may further result in cavitation not being generated at the treatment location according to the drive parameters laid out in the treatment plan. As mentioned above, one solution is to simply increase the drive voltage of the transducer array to form cavitation even in the presence of the blockage. However, another approach is to manually or automatically enter a “tilt and translate” configuration with the transducer array to avoid the blockage and maintain a similar acoustic window to the treatment location. To determine the parameters for tilting and translating the therapy transducer array to avoid or minimize a blockage, the electronic controller and/or the robotic positioning system/arm can be configured to perform the following steps:

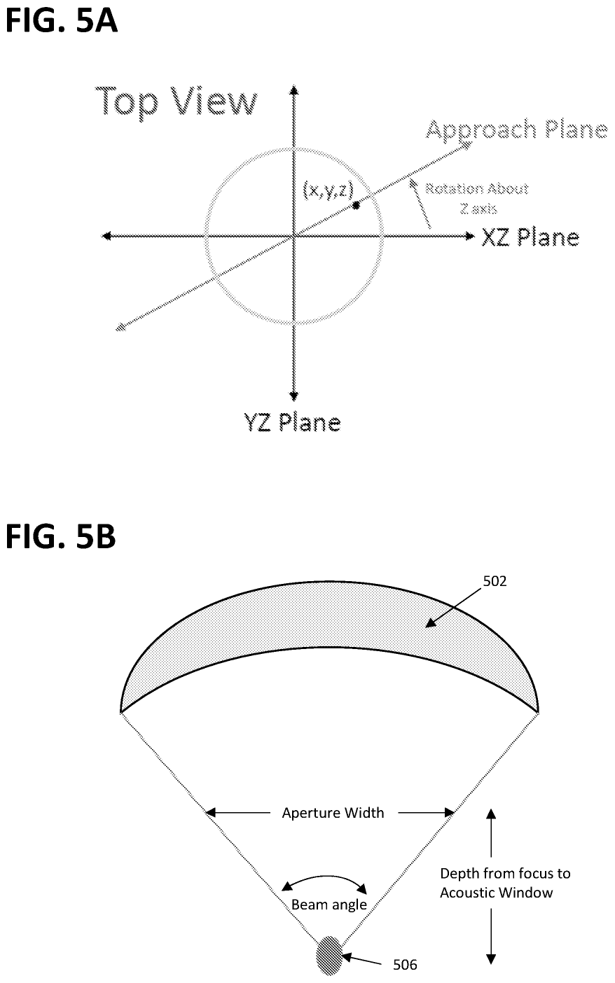

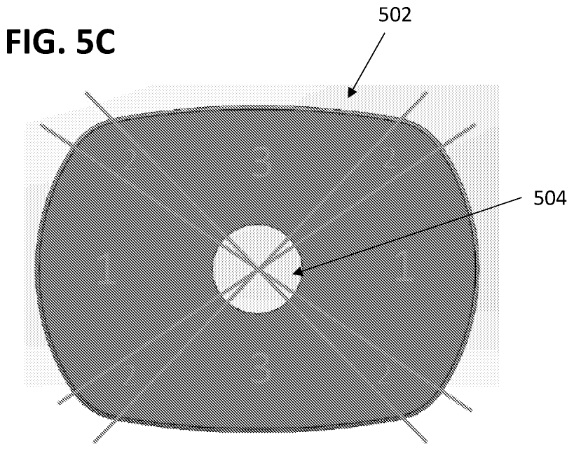

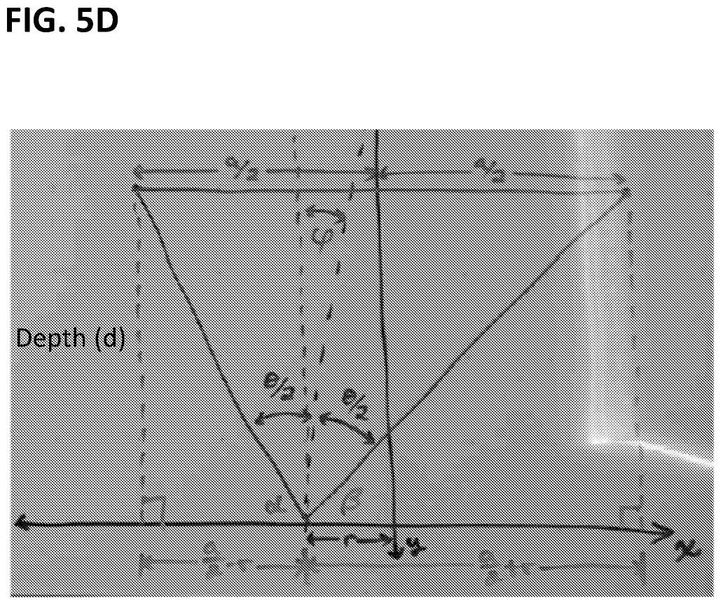

Step 1) Determine the approach plane. Referring to FIG. 5A, a top-down view of a target tissue volume is provided. In some embodiments, processors and/or electronic controllers of the system and/or the robotic positioning system/arm can be configured to automatically determine an approach plane for a given treatment location (x, y, z) in a target tissue volume. An ‘Approach Plane’ is a plane, starting from the XZ plane and rotated about the Z axis that contains the treatment location (x, y, z).

Step 2) Calculate the aperture width at the acoustic window. The aperture width of the transducer array for a given acoustic window is a function of the beam angle of the transducer and the depth of the acoustic window. The beam angle of the transducer depends on the specific geometry of the transducer. The aperture width at the acoustic window defines the region through which ultrasound energy is delivered or transmitted by the transducer array. In order to determine the parameters for tilting and/or translating the therapy transducer, the aperture width at the desired acoustic window depth must be calculated. This can be accomplished by calculating the transducer beam angle in the approach plane for the specific transducer geometry.

In some embodiments, the beam angle for a given transducer array is known. Therefore, the aperture width at a given acoustic window depth can be calculated based on the beam angle and the distance from the focus to the acoustic window. In other embodiments, it may be necessary to calculate the beam angle for the transducer array. The simplest transducer geometry for determining beam angle is with a circular aperture transducer, which can be determined by using known trigonometric equations for a circular aperture. Similarly, beam angles for ellipsoidal transducer geometries may also be calculated with known trigonometric equations. Additionally, other equations may be used to calculate the beam angle for other aperture geometries.

More complicated transducer geometries may require dividing the transducer surface into sections for which separate trigonometric equations can be used to determine the overall beam angle. For example, referring to FIG. 5C, a bottom-up view of a therapy transducer array is provided. As shown, FIG. 5C includes a therapy transducer array 502 and an aperture 504 in the array for an imaging system, such as an ultrasound probe. It should be understood that in some embodiments, there is no aperture or imaging probe located in the center of the therapy transducer array. The transducer array 502 comprises a complex geometry that includes a cropped elliptical shape with “fillets”, wherein the fillets (region 2) refer to a specific corner blending technique in the transducer design to transition between the cropped portion of the transducer aperture (region 3) and the circular portion of the transducer aperture (region 1). More specifically, the apertures shown in the embodiment of FIG. 5C include round sections (region 1), elliptically cropped sections (region 3), and corner interface sections (region 2) between the round and elliptically cropped regions filleted to remove sharp corners. The beam angle in the approach plane for a given treatment point/location must be calculated for these specific regions using trigonometric equations.

Referring still to FIG. 5C, regions 1 represent the beam angle to be calculated of a circular aperture transducer. Regions 2 represent the beam angle to be calculated of “fillets” of an elliptically cropped shaped transducer, and regions 3 represent the beam angle to be calculated of an elliptically cropped shaped transducer. In this example, the approach plane rotation is the input to calculating the beam angle, determining which equation to use to calculate the beam angle.

Step 3) Calculate the tilt angle (φ) to center the beam on the central acoustic window while reaching the target treatment location (x, y, z). In some embodiments, the tilt angle is the angle the transducer array is to be tilted in the approach plane away from the Z axis. FIG. 5D illustrates a schematic view of variables used to determine the tilt angle, including the depth (d) to the acoustic window which can be an input to the system. The tilt angle (φ) can be calculated by the system to center the beam on the central acoustic window while reaching the target treatment location.

In some embodiments, a user of the system can specify the position and/or depth (d) of the acoustic window to be preserved when calculating the tilt angle. In another embodiment, the depth (d) can be automatically determined by the system. This determination can be based, for example, on pre-treatment imaging such as MRI or CT which may identify one or more obstructions in the tissue (e.g., bone or ribs). In some examples, the depth (d) can be set at the actual blockage depth of an aberrator or obstruction, so as to align the acoustic window with potential obstructions in the beam path. Reduced variability in the blockage can result in more consistent cavitation threshold determinations (e.g., during threshold testing).

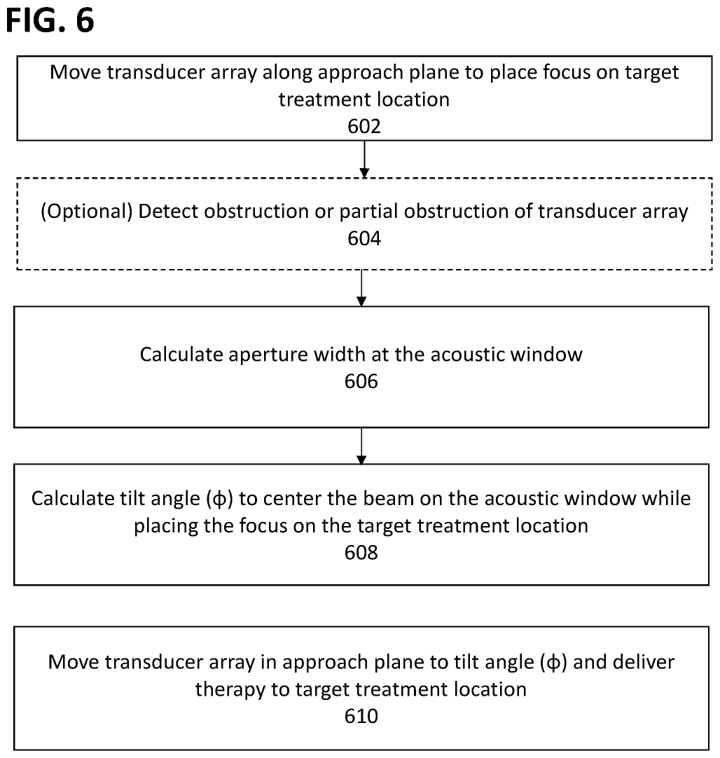

FIG. 6 is a flowchart illustrating the steps described above and in FIGS. 5A-5D. These steps can comprise a method of delivering ultrasound therapy to a patient. Referring to FIG. 6, at step 602, the method can comprise moving a transducer array along an approach plane to place a focus of the transducer array on a target treatment location (x, y, z) within a treatment volume. As described above, a treatment plan can be implemented or calculated that divides a treatment volume into a plurality of treatment locations. The transducer array can then be automatically or robotically controlled to deliver ultrasound/histotripsy therapy subsequently to each of the plurality of treatment locations to complete the treatment according to the treatment plan. Sometimes this treatment plan can be implemented by simply translating the transducer array laterally without tilting so as to treat each treatment location within the treatment plan.

However, referring to step 604, in some embodiments, the system can optionally detect an obstruction or partial obstruction of the transducer array, or alternatively, the user can identify an obstruction or partial obstruction. For example, one or more transducer elements or a portion of the transmit aperture of the transducer array may be blocked by a blockage such as bone or other hard tissues (native or foreign) located between the array and the target treatment location. The obstruction or blockage can be determined, for example, with pre-treatment imaging. In another embodiment, the blockage can be detected by receiving echoes of the transmitted ultrasound signals to determine that a blockage or obstruction is present.

In this embodiment, instead of attempting to deliver ultrasound energy to the treatment location with a partially blocked array, the method can further comprise determining a tilt angle of the transducer array that places the focus of the beam from the transducer array at the target treatment location while reducing or eliminating the number of blocked transducer elements or the size of the blocked aperture of the transducer array.

At step 606, the method can include calculating an aperture width for a given acoustic window. In some examples, the acoustic window is defined as being at a depth (d) from the focus of the transducer array. For example, if bones or other aberrators are identified at a depth (d) from the focus, then the bones or aberrators can be used to define the acoustic window. The aperture width is a function of the beam angle of the transducer array and the depth of the acoustic window. This beam angle can depend on the type of transducer array used. For example, the beam angle for a circular aperture transducer array will be different than the beam angle of an elliptical transducer array, or that of a cropped elliptical transducer array with “fillets”. In some embodiments, the beam angle is known. In other embodiments, the system or the user can calculate the beam angle so as to determine the aperture width at the acoustic window.

Next, at step 608, the method can include calculating a tilt angle (φ) to center the beam on the acoustic window while keeping the focus of the transducer array on the target treatment location.

At step 610, a robotic positioning system can be configured to control or adjust the orientation of the transducer array to the tilt angle from step 608. This allows the transducer array to center the beam on the acoustic window while still allowing the focus of the transducer to be at the target treatment location. As described above, the therapy can comprise delivering histotripsy pulses to generate cavitation in the treatment location. In some embodiments, the tilted approach may require more working distance, so use may be limited for very deep target tissue locations. This may result when using a smaller depth (d) (i.e., when the maximized acoustic window is close to the target). The extent of the tilt may be limited by sphericity of the bubble cloud. For example, in some embodiments, the bubble cloud is not spherical, but instead is elongated or more of an elliptical shape.

The method described above will use tilting of the transducer array instead of translation, causing the transducer array to stay more stationary during treatment. This typically only results when using a larger depth (d) (i.e., when the maximized acoustic window is close to the therapy transducer), allowing less spatial variability in traversal. This can advantageously reduce collisions and enable a smaller coupling container/water bath.

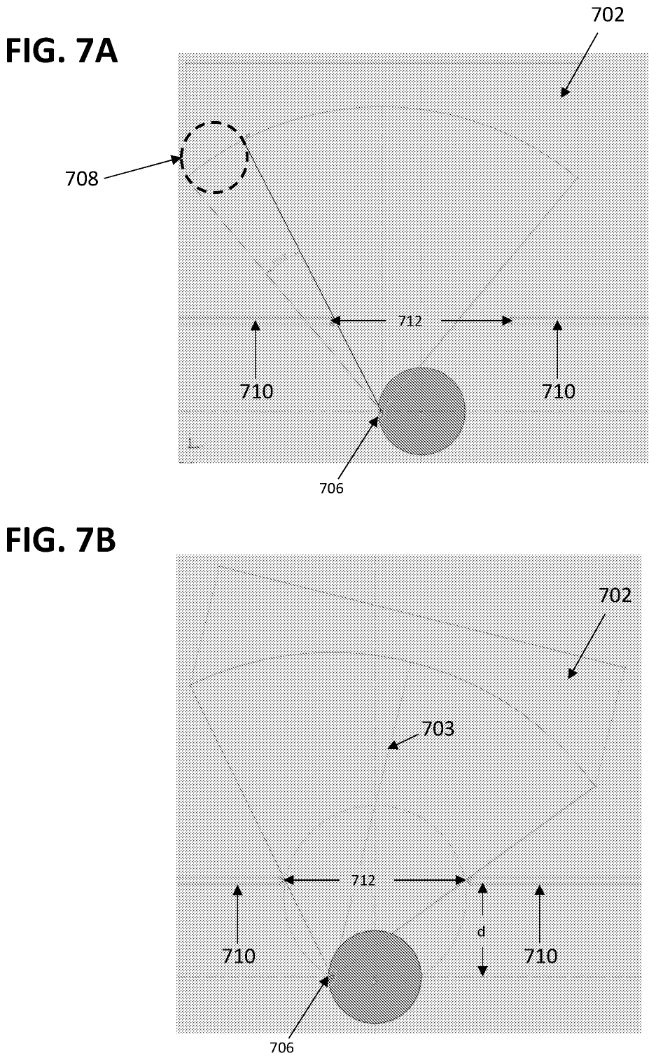

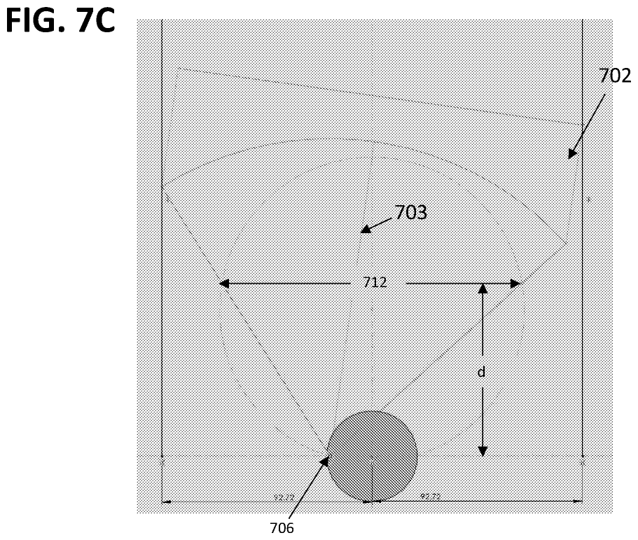

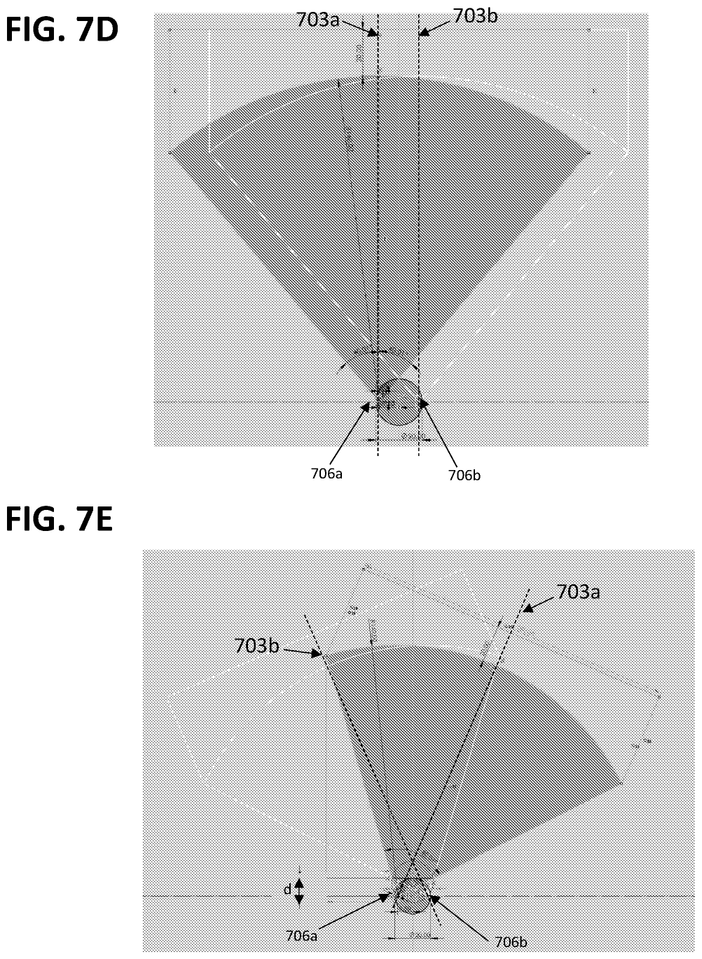

Additional examples are shown in FIGS. 7A-7E showing how changing the depth (d) input between the focus and the desired acoustic window can change the tilt angle and/or the lateral movement of the transducer array for a given treatment volume and target treatment location.

FIG. 7A shows a transducer array 702 with a focus 706 positioned at a lateral extreme of a target tissue volume. As shown, the beam of the transducer angle is passing through an acoustic window 712 defined by one or more bones or aberrators 710 (e.g., ribs). In this example, without tilting the transducer, a portion of the transducer aperture 708 is blocked by the aberrator 710 as shown, preventing that acoustic energy from reaching the focus 706. FIG. 7B shows an embodiment according to the description above in which the depth input is set to an “aberrator” depth setting, which marks the depth at the distance between the focus and the aberrators or bones. At this “aberrator” depth setting, the acoustic window is defined as being between the aberrators at a depth d from the focus to the aberrators. With this acoustic window known, the tilt angle of the transducer can be calculated to center the beam of the transducer at the acoustic window while still keeping the focus of the transducer on the target treatment location. The transducer can be correspondingly tilted to reduce or eliminate any blockage of the transmit aperture, which can therefore eliminate the need to increase the driving voltage to achieve cavitation at the focus.

Adjusting the depth (d) value can provide additional benefits or advantages during therapy. In some embodiments, the depth input can be set to a “high” depth, which places the depth setting closer to the transducer aperture. This enables the transducer to tilt, as opposed to translate, maintaining the transducer in a more stationary position. A “high” depth setting can be used to minimize transducer movement in the lateral direction during a treatment plan. In some embodiments, the “high” depth setting is an optimal depth setting to achieve the least required lateral transducer movement for a given treatment plan. This setting is a function of the therapy transducer array geometry alone, since no user directed or automatic detection of the anatomy is required towards this goal of minimizing transducer array motion. For example, referring to FIG. 7C, the depth value (d) is significantly increased compared to the value shown in FIG. 7B, which increases the aperture width for the new acoustic window (at depth d). This increased depth input can reduce the amount of lateral movement required to reach all the treatment location points within the target tissue volume. For example, with no tilting of the transducer array implemented in the treatment plan, the robotic positioning system may require up to 20-25% more lateral movement. Increased lateral movement requires additional space above the patient, and requires additional acoustic coupling between the transducer array and the patient (e.g., the acoustic coupling container described herein must be 20-25% larger).

In other embodiments, reducing the depth of the acoustic window can result in more or a higher degree of tilting of the transducer array throughout a given treatment plan to reach all the treatment locations. FIG. 7D shows an example of a tissue path of a transducer array between opposite treatment locations 706a and 706b with no tilting implemented. In this example, there is a similar tissue path for opposite treatment points. The therapy transducer array is moved through the target tissue volume, such as between treatment locations 706a and 706b, while maintaining an identical transducer array pose (e.g., vertically aligned along the Z-axis with no tilting between treatment locations). Therefore the pose axis 703a when treating point 706a is the same as the pose axis 703b when treating point 706b. However, in FIG. 7E, with the depth value d set to a low value (e.g., very close to the focus), very different treatment paths and tilt or pose angles are required to treat the entire volume. Thus, while the therapy transducer array is tilted along pose axis 703a (to the right of the page) to treat point 706a, the therapy transducer array is tilted along pose axis 703b (to the left of the page) to treat point 706b. As a result, this spreads the thermal deposition of the ultrasound energy over the course of a treatment plan, resulting in a reduction in “hot spot” formations within the target tissue volume while also reducing the amount of lateral movement of the therapy transducer array to treat the target tissue volume.

As a representative example, a target tissue volume may comprise treating one or more tumors, in one or more segments of the liver, where the acoustic pathway to at least one of the tumors, may include overlying rib and bony obstruction. An example embodiment of the system in this example may be configured with software to allow the user to visualize the target tumor and respective acoustic pathway (e.g., via ultrasound), and a graphical user interface display of the acoustic field (for the respective and representative therapy transducer with its focus positioned on the target tumor), to further allow the user to select the desired 3D trajectory from the skin surface to the target tumor, wherein the selected trajectory contemplates a preferred approach around and/or through the identified obstruction in order to minimize the overlap with the transducer acoustic field and the obstruction.

In another embodiment, the system software and graphical user interface, allows a dynamic visualization and simulation of the therapy transducer acoustic field, the target, planned treatment volume contours (e.g., a displayed shape enveloping the user selected target), and the respective tilt angles and motion pattern for the given combination. The visualization and dynamic display enables the user to assess the selected trajectory and treatment plan prior to accepting the plan and initiating/delivering any therapy pulses (e.g., for bubble cloud calibration, test pulses for establishing voltage settings and/or automated treatment).

In one example, the tilting feature is used to treat a small renal mass in the kidney wherein the acoustic window and pathway to the renal mass includes overlying rib, fascia and adjacent bowel, and where minimizing linear translation of the therapy transducer across the body and over the identified blockers/obstruction is used to reduce energy delivery in/through them.

In another embodiment, the system software includes a pre-planning software that further allows the user to simulate a patient/procedure specific set up, for a respective therapy transducer model, and select a preferred trajectory/approach, and then export the pre-planning file to the system (e.g., therapy system) to position the therapy transducer focus (via the robotic arm) on the user selected tumor/target using the robot pose/position defined in the pre-planning software.

In one example, the tilt feature is implemented on a system configured with an acoustic coupling and patient interface that comprises a coupling frame interfaced to the outer surface of the therapy transducer or treatment head, where the combination of the coupling frame and therapy transducer share the same main axis alignment/trajectory to the user selected target, the coupling frame and therapy transducer are enabled to tilt (and motion) together.

In another example, wherein the tilt embodiment is configured to treat the pancreas and minimize/avoid adjacent organs, bony anatomy and/or bowel structures, and/or specifically defined critical structures (e.g., vascular, biliary, etc.).

In another example, wherein the system is configured to treat through a craniotomy and in some use cases, where the dura may remain intact.

In another example system embodiment, wherein the system displays acoustic field overlays on ultrasound, MRI, CT, PET/CT and/or combinations of, and in some use cases wherein the target tumors, blockers/obstruction, skin surface, body wall and/or adjacent organs/tissues may be visualized, segmented and/or displayed in context to the acoustic field display.

In a related example, wherein the ultrasound is displayed in real-time as rigidly or deformably registered to another imaging modality, including but not limited to, MRI, CT, PET/CT, or other forms of X-ray based images, including fluoroscopic or cone beam CT images.

In another example, wherein the displayed images are acquired with the patient coupling system set up on the patient.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles.

Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below).

Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a sealable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

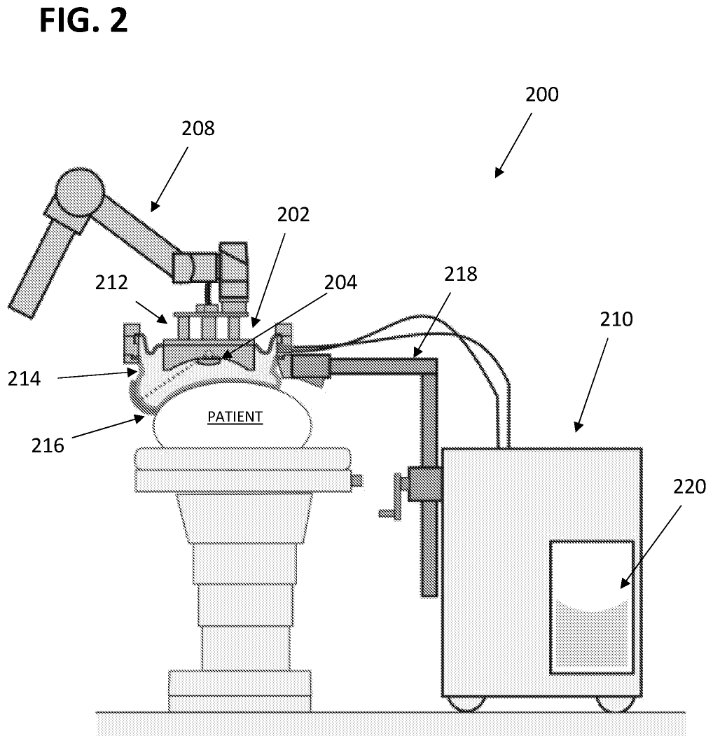

The disclosed histotripsy acoustic and patient coupling systems, in general, may comprise one or more of the following sub-systems and components, an example of which is depicted in FIG. 2, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

FIG. 2 illustrates one embodiment of a histotripsy therapy and imaging system 200, including a coupling assembly 212. As described above, a histotripsy therapy and imaging system can include a therapy transducer 202, an imaging system 204, a robotic positioning arm 208, and a cart 210.

The therapy and/or imaging transducers can be housed in a coupling assembly 212 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 212 is supported by a mechanical support arm 218 which can be load bearing in the x-y plane but allow for manual or automated z-axis adjustment. The mechanical support arm can be attached to the floor, the patient table, or the cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The system can further include a fluidics system 220 that can include a fluid source, a cooling and degassing system, and a programmable control system. The fluidics system is configured for external loading of the coupling membrane with automated control of fluidic sequences. Further details on the fluidics system 220 are provided below.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the UMC can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “UMC” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the UMC/coupling solution. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium, where preparation may include the ability to degass, chill, monitor, adjust, dispense/fill, and retrieve/drain coupling medium to/from the frame/assembly. The fluidics system may include an emergency high flow rate system for rapid draining of the coupling medium from the UMC. In some embodiments, the fluidics system can be configured for a single use of the coupling medium, or alternatively, for re-use of the medium. In some embodiments, the fluidics system can implement positive air pressure or vacuum to carry out leak tests of the UMC and membrane prior to filling with a coupling medium. Vacuum assist can also be used for removal of air from the UMC during the filling process. The fluidics system can further include filters configured to prevent particulate contamination from reaching the UMC.

The fluidics system may be implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

1.-20. (canceled)

21. A method of providing ultrasound therapy, comprising:
moving an ultrasound transducer array of an ultrasound system along an approach plane to place a focus of the ultrasound transducer array on a target treatment location within a treatment volume of a subject;
determining an aperture width of the ultrasound transducer array in the approach plane at a depth of an acoustic window;
determining a tilt angle that centers an acoustic beam of the ultrasound transducer array on the selected acoustic window while placing the focus of the ultrasound transducer array on the target treatment location;
controlling movement of the ultrasound transducer with a robotic positioning system along the approach plane to assume the tilt angle that places the focus on the target treatment location and centers the acoustic beam on the selected acoustic window; and
delivering ultrasound therapy to the target treatment location with the ultrasound transducer array.

22. The method of claim 21, wherein the ultrasound therapy comprises histotripsy therapy.

23. The method of claim 21, wherein delivering ultrasound therapy further comprises generating cavitation at the target treatment location.

24. The method of claim 21, wherein determining the aperture width further comprises calculating a beam angle of the ultrasound transducer array in the approach plane.

25. The method of claim 24, wherein determining the beam angle is based on the specific ultrasound transducer geometry.

26. The method of claim 21, wherein the ultrasound transducer geometry comprises a circular aperture.

27. The method of claim 21, wherein the ultrasound transducer geometry comprises an ellipsoidal aperture.

28. The method of claim 21, wherein the ultrasound transducer geometry comprises a cropped elliptical shape.

29. The method of claim 21, wherein the ultrasound transducer geometry comprises cropped elliptical regions, spherical regions, and fillet regions that transition between the cropped elliptical regions and the spherical regions.

30. The method of claim 21, wherein the approach plane is rotated around a z-axis of the transducer array.

31. The method of claim 21, wherein the depth is user-selected.

32. The method of claim 21, wherein the depth is automatically selected.

33. The method of claim 21, wherein the depth is a distance from the target treatment location to an obstruction.

34. The method of claim 33, wherein the obstruction comprises one or more bones in the subject.

35. The method of claim 34, wherein the one or more bones define the acoustic window.

36. The method of claim 21, wherein the tilt angle reduces or eliminates obstruction of at least a portion of a transmit aperture of the ultrasound transducer array.

37. The method of claim 21, wherein the tilt angle is determined with one or more processors of the ultrasound system.

38.-49. (canceled)

50. An ultrasound system, comprising:
a robotic positioning system;
an ultrasound transducer array disposed on the robotic positioning system, the ultrasound transducer array being configured to deliver ultrasound pulses into a subject;
one or more processors operatively coupled to the robotic positioning system and the ultrasound transducer array;
a non-transitory computing device readable medium having instructions stored thereon for generating a treatment plan for ultrasound therapy, wherein the instructions are executable by the one or more processors to cause the ultrasound system to:
move the ultrasound transducer array along an approach plane to place a focus of the ultrasound transducer array on a target treatment location within a treatment volume of the subject;
determine an aperture width of the ultrasound transducer array in the approach plane at a depth of an acoustic window; and
determine a tilt angle of the ultrasound transducer array that centers an acoustic beam of the ultrasound transducer array on the acoustic window while placing the focus of the ultrasound transducer array on the target treatment location.

51. The ultrasound system of claim 50, wherein the instructions are further configured to cause the ultrasound system to:
control the robotic positioning system to orient the ultrasound transducer array at the tilt angle while maintaining the focus at the target treatment location; and
deliver ultrasound therapy to the target treatment location with the ultrasound transducer array.

52. The system of claim 50, wherein the ultrasound system is configured to determine the aperture width by calculating a beam angle of the ultrasound transducer array in the approach plane.

53.-69. (canceled)
